(19) United States
(12) Patent Application Publication
(10) Pub. No.: US 2004/0101917 A1
(43) Pub. Date: May 27, 2004

(54) Methods for managing animal processing operations

(76) Inventors: W. Wade Robey, Excelsior, MN (US); Alison M. Jones, Eden Prairie, MN (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/394,763

(22) Filed: Mar. 21, 2003

Related U.S. Application Data
(63) Continuation-in-part of application No. 10/306,113, filed on Nov. 27, 2002.

(51) Int. Cl. G01N 33/554; G01N 33/569; G06F 19/00; G06G 7/58; G01N 33/48; G01N 33/50; C12Q 1/04

(52) U.S. Cl. 435/7.32; 435/34; 702/19

(57) ABSTRACT

Methods for managing animal processing operations are described that include scheduling animals for processing according to risk of contamination.

### CROSS-REFERENCE TO RELATED APPLICATIONS

This application is a continuation-in-part of U.S. Ser. No. 10/306,113, filed Nov. 27, 2002.

### TECHNICAL FIELD

This invention relates generally to animal processing, and more particularly to scheduling processing of animals based at least in part on microbial loads and/or microbial profiles of the animals.

### BACKGROUND

Food borne diseases cause an estimated 76 million illnesses and 5,000 deaths each year in the United States, with direct and indirect medical costs estimated to be $1 billion yearly. Mead et al. Emerg. Infect. Dis., 5(5):607-625 (1999). Common food pathogens include Salmonella, Listeria monocytogenes, Escherichia coli, Campylobacter jejuni, and Bacillus cereus. Outbreaks of food borne diseases typically are associated with contaminated meat products, raw milk, and eggs. Post-harvest intervention strategies (e.g., pasteurization by heat, irradiation, etc.) during the processing of food products could significantly reduce the risk of transmission of pathogens from farm to table. Cost and consumer acceptance issues, however, have delayed application. Thus, a need exists for pre-harvest and in-harvest intervention strategies to effectively reduce human food-borne illness.

### SUMMARY

The invention is based on methods for managing animal processing operations that include determining microbial loads and/or microbial profiles of animals to be processed and scheduling processing of the animals according to the risk of contamination. For example, higher risk animals (e.g., high microbial load and/or incidence of particular pathogens) can be scheduled for processing on the same day. Alternatively, lower risk animals (e.g., a low microbial load and lower incidence of particular pathogens) can be scheduled for processing earlier in the day and higher risk animals can be scheduled for processing later in the day. Such scheduling minimizes contamination during the processing.

In one aspect, the invention features a method of managing an animal processing operation. The method includes providing biological samples from a plurality of animals; determining microbial load (e.g., total number of enteric bacteria) of each biological sample; and scheduling processing of the plurality of animals based at least in part on the microbial load. The microbes can be bacteria, protozoa, or fungi. The animals can be birds such as chickens or turkeys, pigs, cows, or fish. In some embodiments, the plurality of animals includes a plurality of different populations of animals. Scheduling can include ranking the animals from lowest microbial load to highest microbial load and integrating the ranking with one or more factors selected from the group consisting of number of animals to be processed, weight targets of animals, proximity of animals to animal processing facility, and time of delivery of animals to animal processing facility.

The method further can include determining the microbial profile of each biological sample and scheduling processing of the plurality of animals based at least in part on the microbial load and microbial profile. Scheduling can include ranking the animals from lowest risk to highest risk based on the microbial load and microbial profile to provide a risk profile; and integrating the risk profile with one or more factors selected from the group consisting of number of animals to be processed, weight targets of animals, proximity of animals to animal processing facility, and time of delivery of animals to animal processing facility. Determining the microbial profile can include taxonomic and phylogenetic identification, histological analysis, immunological analysis, genetic fingerprinting, ribosomal genotyping, or cpn60 genotyping.

The biological samples can be gastrointestinal tract samples such as digesta, mucous, mucosal tissue, and feces; periodontal samples; or bodily fluids such as blood, urine, saliva, sputum, or semen. In some embodiments, the biological samples include pooled samples from each population of animals The invention also features a computer-readable storage medium having instructions stored thereon for causing a programmable processor to rank a plurality of animals from lowest microbial load to highest microbial load. The computer-readable storage medium further can include instructions for scheduling processing of the plurality of animals based at least in part on the rank. The computer readable storage medium also can include instructions for scheduling processing of the plurality of animals based on the rank and one or more factors selected from the group consisting of number of animals to be processed, weight targets of animals, proximity of animals to animal processing facility, and time of delivery of animals to animal processing facility.

In some embodiments, the computer-readable storage medium further includes instructions for causing the programmable processor to determine a risk profile for the plurality of animals based on microbial loads and microbial profiles from the plurality of animals and to rank the plurality of animals from lowest risk to highest risk. Such a computer-readable storage medium further can include instructions for scheduling processing of the plurality of animals based on the risk profile and one or more factors selected from the group consisting of number of animals to be processed, weight targets of animals, amount of feed, proximity of animals to animal processing facility, and time of delivery of animals to animal processing facility.

In another aspect, the invention features a system for scheduling a plurality of animals for processing. The system includes means for inputting microbial loads of the plurality of animals; means for inputting microbial profiles of the plurality of animals; and a processor for correlating microbial load and microbial profiles with risk and scheduling the plurality of animals for processing based at least in part on risk.

The invention also features a method of managing an animal processing operation. The method includes providing a risk profile of a plurality of animals; and communicating the risk profile to a manager of the animal processing operation.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DESCRIPTION OF DRAWINGS

FIG. 1 is the sequence of a cpn60 gene from Clostridium perfringens (SEQ ID NO: 1; GenBank® Accession No. NC_{—}003366). Sequences to which the universal cpn60 primers described herein can hybridize (or the complement thereof) are underlined.

FIG. 2 is the sequence of a cpn60 gene from Escherichia coli (SEQ ID NO:2; GenBank Accession No. NC_{—}000913). Sequences to which the universal cpn60 primers described herein can hybridize (or the complement thereof) are underlined.

FIG. 3 is the sequence of a cpn60 gene from Staphylococcus coelicolor (SEQ ID NO:3; GenBank Accession No. AL939121). Sequences to which the universal cpn60 primers described herein can hybridize (or the complement thereof) are underlined.

FIG. 4 is the sequence of a cpn60 gene from Campylobacter jejuni (SEQ ID NO:4; GenBank® Accession No. NC_{—}002163). Sequences to which the universal cpn60 primers described herein can hybridize (or the complement thereof) are underlined.

FIG. 5 is the sequence of a cpn60 gene from Salmonella enterica (SEQ ID NO:5; GenBank® Accession No. NC_{—}003198). Sequences to which the universal cpn60 primers described herein can hybridize (or the complement thereof) are underlined.

### DETAILED DESCRIPTION

In general, the invention provides methods that allow animal processing facilities (e.g., abattoirs or slaughter-houses) to schedule processing of animals according to risk of contamination, which can mitigate potential food safety problems. Methods of the invention can be used for any farmed animal that will be subjected to mass processing (e.g., birds such as chickens and turkeys, pigs, cows, or fish). Risk of contamination is assessed based on the microbial load (i.e., quantity of microbes) and/or microbial profile (i.e., identification of individual strains, subspecies, species, and/or genera of microorganisms within a community of microorganisms). The ability to rapidly determine the quantity and presence of particular pathogens is particularly useful in scheduling processing of animals.

Methods of Managing Animal Processing Operations

In general, biological samples are provided from a plurality of animals and microbial loads and/or microbial profiles are determined from each sample. Biological samples can be obtained from each animal to be processed, or alternatively, obtained from a representative number of animals within a population (e.g., farms, trucks, herds, houses, pens, or feedlots). Biological samples obtained from the same population may be pooled. For example, on a farm containing three houses of chickens, a representative number of biological samples can be obtained from each house and the samples from each house pooled together and analyzed.

Biological samples can be any sample obtained directly from an animal, or any sample that has been in contact with such an animal, and from which microbes or microbial nucleic acid can be isolated. Representative biological samples that can be obtained directly from an animal include samples obtained from the gastrointestinal tract (GIT) (e.g., digesta, mucous and/or mucosal tissue, or feces), from the mouth or teeth (i.e., a periodontal sample), or from bodily fluid such as blood, urine, saliva, sputum, or semen. Such biological samples can be obtained from an animal using methods and techniques known in the art. See, for example, Diagnostic Molecular Microbiology: Principles and Applications (Persing et al. (eds), 1993, American Society for Microbiology, Washington D.C).

Additionally, a biological sample suitable for use in the methods of the invention can be a substance that one or more animals have contacted. For example, a soil sample that one or more animals have contacted, or, for example, litter on which an animal has deposited fecal or other biological material can be used in the methods of the invention. Nucleic acids can be isolated from such biological samples using methods and techniques known in the art. See, for example, Diagnostic Molecular Microbiology: Principles and Applications (Persing et al. (eds), 1993, American Society for Microbiology, Washington D.C).

In some embodiments, a separation and/or concentration step may be necessary to separate any microbes present from other components of a biological sample or to concentrate the microbe to an amount sufficient for rapid detection. For example, a sample suspected of containing a biological microbe may require a selective enrichment of the microbe (e.g., by culturing in appropriate media for a sufficient amount of time, such as for 4-96 hours, or longer) prior to detecting the microbial profile. Alternatively, appropriate filters and/or immunomagnetic separations can concentrate microbes without the need for an extended growth stage. For example, antibodies having specific binding affinity for microbial surface antigens can be attached to magnetic beads and/or particles and used to isolate microbes. Polyclonal or monoclonal antibodies can be used to isolate microbes. Polyclonal antibodies are heterogenous populations of antibody molecules that are specific for a particular antigen and contained in the sera of immunized animals. Monoclonal antibodies are homogeneous populations of antibodies to a particular epitope contained within an antigen. Antibody fragments that have specific binding affinity for microbial surface antigens also can be used. Such fragments include, but are not limited to, F(ab′)₂ fragments that can be produced by pepsin digestion of the antibody molecule, and Fab fragments that can be generated by reducing the disulfide bridges of F(ab′)₂ fragments. Alternatively, Fab expression libraries can be constructed. See, for example, Huse et al., 1989, Science, 246:1275. Single chain Fv antibody fragments are formed by linking the heavy and light chain fragments of the Fv region via an amino acid bridge (e.g., 15 to 18 amino acids), resulting in a single chain polypeptide. Single chain Fv antibody fragments can be produced through standard techniques. See, for example, U.S. Pat. No. 4,946,778.

Multiplexed separations in which two or more concentration processes are employed also can used, including combinations of two or more of the following techniques: centrifugation, membrane filtration, electrophoresis, ion exchange, affinity chromatography, and immunomagnetic separations.

Determining Microbial Load and Profile

Microbial loads and/or profiles of animals can be determined on-site at an animal processing facility or off-site from the processing operation (e.g., at a farm). Microbial load, which refers to the quantity of microbes, can be determined using known microbiological techniques including aerobic plate counts (e.g., 3M Petrifilm™ or Redigel™ products, or IDEXX Simplate total plate count products). In some embodiments, microbial load can be total number of enteric bacteria.

As discussed above, microbial profiles are representations of individual strains, subspecies, species, and/or genera of microorganisms within a community of microorganisms. Generally, determining a microbial profile involves taxonomic and/or phylogenetic identification of the microbes in a community. A microbial profile also can include quantitative information about one or more members of the community. Once one or more microorganisms have been identified in a microbial community, microbial profiles can be presented as, for example, lists of microorganisms, graphical or tabular representations of the presence and/or numbers of microorganisms, or any other appropriate representation of the diversity and/or population levels of the microorganisms in a community.

As used herein, “microbes” refer to bacteria, protozoa, fungi, and viruses. Microbial communities for which a microbial profile can be generated includes, for example, the prokaryotic genera: Staphylococcus, Pseudomonas, Escherichia, Bacillus, Salmonella, Bifidobacterium, Enterococcus, Clostridium, and Campylobacter; protozoa genera: Acanthamoeba, Cryptosporidium, and Tetrahymena; fungal genera: Aspergillus, Candida, and Saccharomyces; and the following viral genus: Coronaviridae.

The microbial profile that is examined can be tailored to the animal to be processed. For example, for poultry, cattle, or swine processing, the presence and/or amount of Campylobacter (e.g., C. jejuni), Listeria (e.g., L. monocytogenes), Escherichia coli, or Salmonella, or combinations thereof, can be examined for the microbial profile. In some embodiments, the microbial profile also can include the presence and/or amount of Clostridium (e.g., C. perfringens or C. botulinum), Staphyococcus (e.g., S. aureus), or both Clostridium and Staphylococcus.

A microbial profile in an animal can be determined using a number of methods. For example, the microbes in a sample can be cultured and colonies identified and/or enumerated. It has been estimated, however, that culturing typically recovers only about 0.1% of the microbial species in a sample (based on comparisons between direct microscopic counts and recovered colony-forming units). An improvement on culture-based methods is a community-level physiological profile. Such determinations can be accomplished by monitoring the capacity of a microbial community to utilize a suite of carbon sources with subsequent detection of the end product of this carbon metabolism by, for example, reduction of a tetrazolium dye. Profiling the physiology of a microbial community can yield qualitative (e.g., different patterns of reduced substrates) and semi-quantitative (e.g., spectrophotometric measurement of reduction) results. Biolog, Inc. (Hayward, Calif.) has commercialized a microtiter plate assay useful for determining the physiological profile of a complex microbial community. The BIOLOG method requires a standard inoculum density of metabolically active microorganisms, and assumes that all members of the community grow at the same rate so the utilization profile is not skewed by the metabolic capabilities of the fastest growers, and further assumes that the 95 substrates reflect the comprehensive substrate availability in the environment of interest.

Culture-independent methods to determine microbial profiles consist of extracting and analyzing microbial macromolecules from a sample. In general, useful target molecules are ones that, as a class, are found in all microorganisms, but are diverse in their structures, thereby reflecting the diversity of the microbes. Examples of target molecules include phospholipid fatty acids (PLFA), polypeptides, and nucleic acids. PLFA analysis is based on the universal presence of modified fatty acids in microbial membranes, and is useful as a taxonomic tool. PLFAs are easily extracted from samples, and separation of the various signature structures reveals the presence and abundance of classes of microbes. This method requires appropriate signature molecules, which often are not known or may not be available for the microbes of interest. In addition, the method requires that an organism's PLFA content does not change under different metabolic conditions. Another limitation to using PLFAs as target molecules is that widely divergent organisms may have the same signature set of PLFAs.

Various nucleic acid-based assays can be employed to determine microbial load and/or microbial profile for animals. The term “nucleic acid” as used herein encompasses both RNA and DNA, including genomic DNA. A nucleic acid can be double-stranded or single-stranded. The choice of target nucleic acid sequence to use for quantifying a microbial organism depends on whether the sequences provide both broad coverage and discriminatory power. Ideally, the target should be present in all members of a given microbial community and be amplified from each member with equal efficiency using common primers, yet have distinct sequences.

Certain nucleic acid-based population methods use denaturation and reannealing kinetics to derive an indirect estimate of the percent (%) guanine and cytosine nucleotides (G+C) content of the DNA in the sample. This method has been used to characterize the total bacterial community in the ileum and cecum of the GIT in poultry, and to examine how diet and other variables modulate the microbial communities in the GITs of animals (Apajalahti et al., 2001, Appl. Environ. Microbiol., 67:5656-67). The % G+C technique provides an overall view of the microbial community and is sensitive only to massive changes in the make-up of the community.

Genetic fingerprinting of a sample from an animal is another method that can be used to determine a microbial profile for an animal. Genetic fingerprinting utilizes oligonucleotide primers with random sequences that hybridize with sequence-specificity to random sequences throughout the genome. Amplification results in a multitude of products. The distribution of amplification products is referred to as a genetic fingerprint. Particular patterns can be associated with a community of microbes in the sample. Genetic fingerprinting, however, lacks the ability to conclusively identify specific microbial species.

Denaturing or temperature gradient gel electrophoresis (DGGE or TGGE) is another technique that can be used to determine a microbial profile for an animal. As amplification products are electrophoresed in gradients with increasing denaturant or temperature, the double-stranded molecule melts and its mobility is reduced. The melting behavior is determined by the nucleotide sequence, and unique sequences will resolve into individual bands. Thus, a D/TGGE gel yields a genetic fingerprint characteristic of the microbial community, and the relative intensity of each band reflects the abundance of the corresponding microorganism. An alternative format includes single-stranded conformation polymorphism (SSCP). SSCP relies on the same physical basis as % G+C renaturation methods, but reflects a significant improvement over such methods.

In addition, a microbial profile for an animal can be determined using terminal restriction fragment length polymorphism (TRFLP). Amplification products can be analyzed for the presence of known sequence motifs using restriction endonucleases that recognize and cleave double-stranded nucleic acids at these motifs. For example, the enzyme Hhal cuts at 5′-GCGC-3′ sites. Using a fluorescently-labeled primer to tag one end of the amplification product and Hhal to digest the products, resolution of this mixture by electrophoresis will yield a series of fluorescent bands whose lengths are determined by how far a 5′-GCGC-3′ motif lies from the terminal tag. TRFLP profiles can be generated using a variety of restriction enzymes, and can be correlated with changes in the population. For example, a TRFLP database for 16S rRNA sequences has been set up at Michigan State University to allow researchers to design experimental parameters (e.g., choice of enzyme and primer combinations). The principal advantages of TRFLP are its robustness and its low cost. Unlike D/TGGE, experimental conditions need not be stringently controlled since the profiles are size-based and thus can be generated by a variety of gel systems, including automated DNA sequencing machines. Alternative approaches include “amplified ribosomal DNA restriction analysis (AADRA)” in which the entire amplification product, rather than just the terminal fragment, is considered. AADRA, however, becomes unmanageable with communities containing many species.

Genotyping of ribosomal DNA (rDNA) (e.g., 16S rDNA) is another way to determine a microbial profile for an animal. 16S rDNA sequences are universal, composed both of highly conserved regions and variable open reading frame (ORF) regions, which allows for the design of common amplification primers and phylogenetic differentiation. 16S ribosomal sequences are relatively abundant in the RNA form. In addition to amplification using oligonucleotide primers, genotyping of 16S rDNA can be performed using other methods including restriction fragment length polymorphism (RFLP) with Southern blotting.

Other targets for genotyping include genes encoding components of RNA polymerase, translation elongation factors, gyrase, and chaperoning, proteins that generally are required for proper folding of polypeptides in vivo. Such protein-encoding sequences may evolve more rapidly than those encoding structural RNAs. Thus, the sequences of protein-encoding sequences in closely related species may have diverged more in closely related species and may provide more discriminatory information. The choice of which target sequence to use depends on whether the sequences provide both broad coverage and discriminatory power. Ideally, the target should be present in all members of a given microbial community, be amplified from each member with equal efficiency using common primers, yet have distinct sequences. Multiple targets may in fact prove necessary for particular applications. Nucleic acid based methods of determining microbial load and/or microbial profile can include an amplification step using, for example, the polymerase chain reaction (PCR).

Nucleic acid hybridization also can be included in determining a microbial profile. Probing amplification products with species-specific hybridization probes is one of the most powerful analytical tools available for profiling. The physical matrix for hybridization can be a nylon membrane (e.g., a macroarray) or a microarray (e.g., a microchip), incorporation of one or more hybridization probes into an amplification reaction (e.g., TaqMan® or Molecular Beacon technology), solution-based methods (e.g., ORIGEN technology), or any one of numerous approaches devised for clinical diagnostics. Probes can be designed to preferentially hybridize to amplification products from individual species or to discriminate species phylogenetically. Probes designed to hybridize to nucleotide sequences from more than one species are referred to herein as “universal probes.”

Conventional PCR techniques are disclosed in U.S. Pat. Nos. 4,683,202, 4,683,195, 4,800,159, and 4,965,188. See also, for example, PCR Primer: A Laboratory Manual, Dieffenbach and Dveksler (eds.), Cold Spring Harbor Laboratory Press, 1995) for standard PCR conditions. PCR typically employs two oligonucleotide primers that bind to a selected nucleic acid template (e.g., DNA or RNA, including messenger RNA). Template nucleic acid need not be purified; it can be a minor fraction of a complex mixture, such as microbial nucleic acid contained in animal cells. Template DNA or RNA can be extracted from a biological or non-biological sample using routine techniques such as those described in Diagnostic Molecular Microbiology: Principles and Applications (supra). Oligonucleotide primers generally are 15 to 50 nucleotides in length (e.g., 15, 16, 17, 18, 19, 20, 21, 22, 23, 24, 25, 26, 27, 28, 29, 30, 35, 40, or 45 nucleotides in length). A primer can be purified from a restriction digest by conventional methods, or can be chemically synthesized. Primers typically are single-stranded for maximum efficiency in amplification, but a primer can be double-stranded. Double-stranded primers are first denatured (e.g., treated with heat) to separate the strands before use in amplification. Primers can be species-specific, i.e., designed to amplify a nucleotide sequence from a particular microbial species, or can be universal, i.e., designed to amplify a sequence from more than one species.

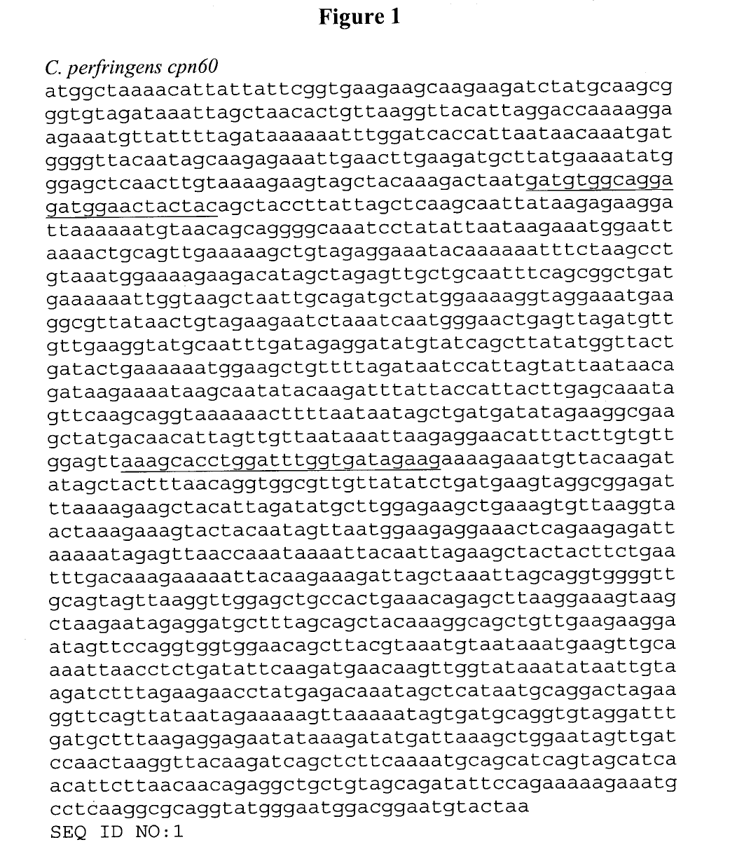

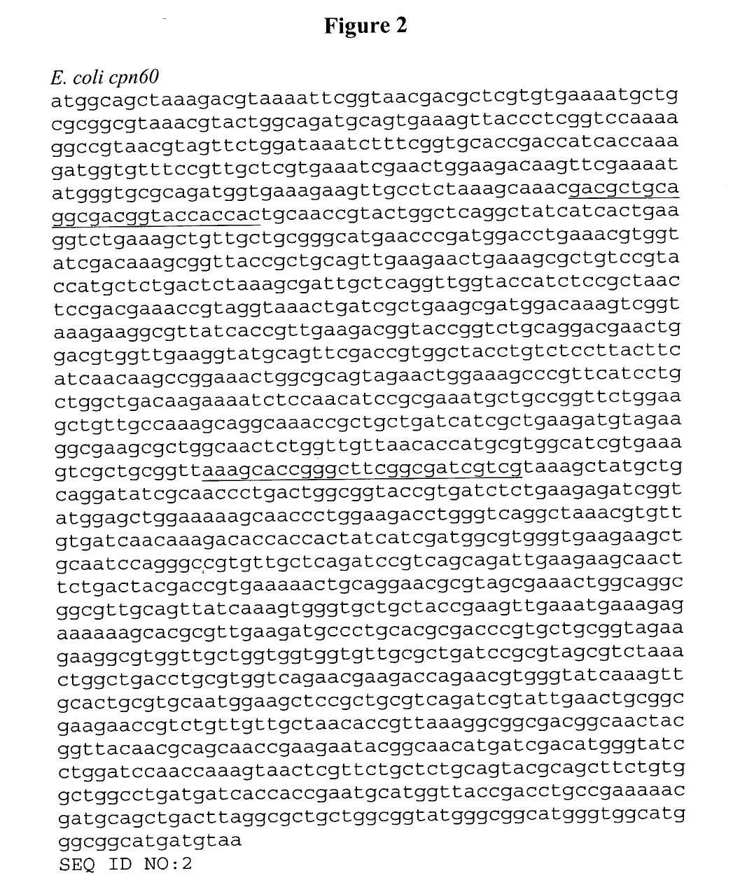

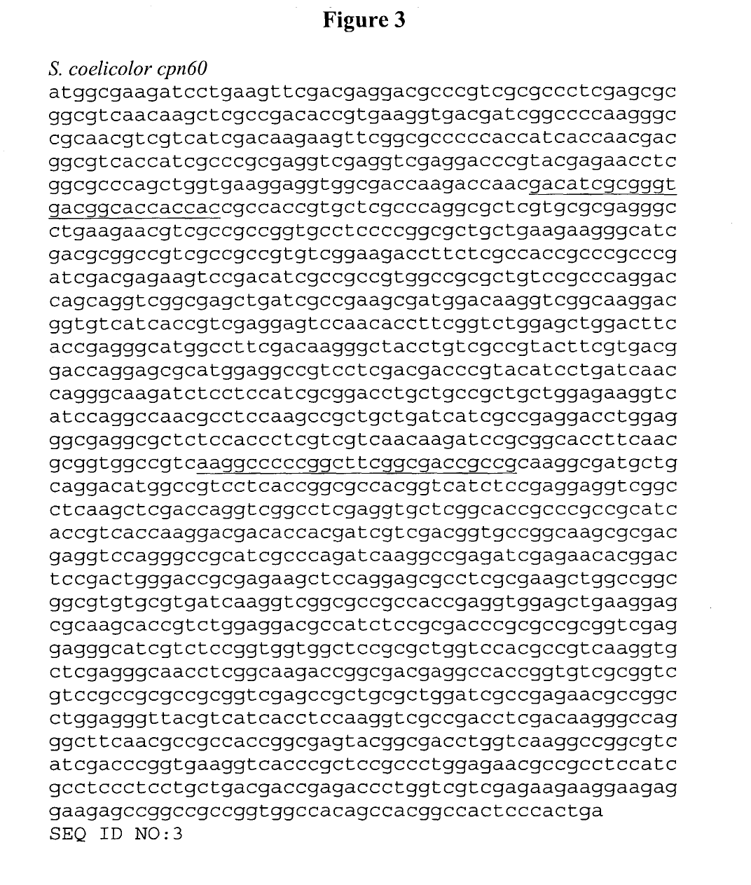

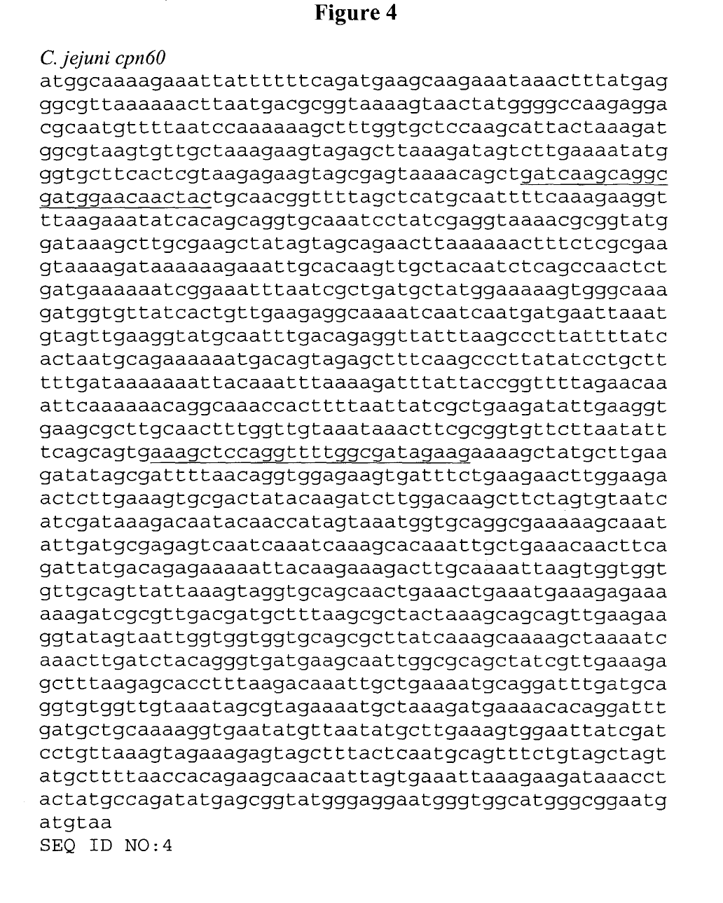

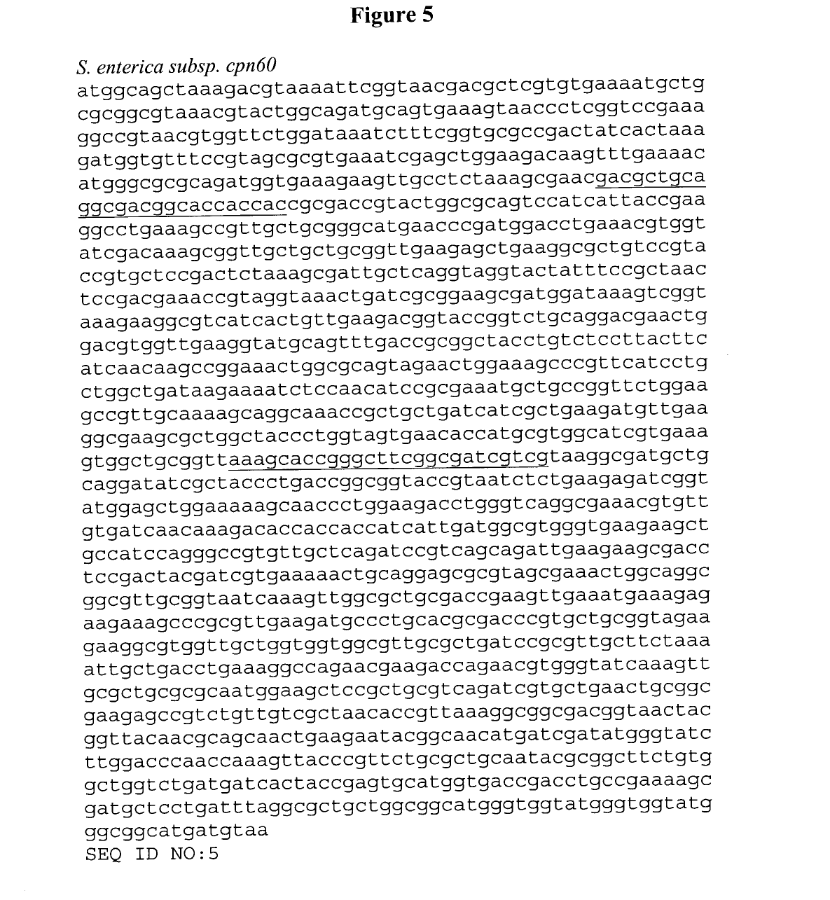

cpn60 nucleic acids are particularly useful nucleic acid targets. cpn60 (also known as hsp60 or GroEL) encodes chaperonin protein 60, and is found universally in prokaryotes and in the organelles of eukaryotes. Sequence diversity within this gene appears greater between and within bacterial genera than for 16S rDNA sequences, making cpn60 particularly useful for determining microbial profiles. Sequences of cpn60 nucleic acids from many microbes are available (see, for example, GenBank Accession Nos. NC_{—}003366, NC_{—}000913, AL939121, NC_{—}002163, and NC_{—}003198; FIGS. 1-5, SEQ ID NOS:1-5, respectively). See also, U.S. Pat. No. 6,497,880, which describes the sequences of Aspergillus fumigatus cpn60 and Candida glabrata cpn60.

PCR oligonucleotide primers (SEQ ID NOS:6 and 7) that universally amplify a 552-558 base pair (bp) segment of cpn60 from numerous microorganisms have been generated (see, for example, U.S. Pat. Nos. 5,708,160 and 5,989,821), and the nucleotide sequences of the amplified cpn60 segments have been evaluated as a tool for microbial analysis. Species-specific primers also can be generated. Primers specific to cpn60 sequences from other microbial organisms can readily be generated by one of ordinary skill in the art. For example, cpn60 nucleotide sequences from two or more microbial species can be aligned to identify variable regions (i.e., regions in which the nucleotide sequences vary between species), and primers can be prepared that hybridize to these regions.

Once a PCR amplification product is generated, it can be detected by, for example, hybridization using FRET technology. FRET technology (see, for example, U.S. Pat. Nos. 4,996,143, 5,565,322, 5,849,489, and 6,162,603) is based on the concept that when a donor fluorescent moiety and a corresponding acceptor fluorescent moiety are positioned within a certain distance of each other, energy transfer taking place between the two fluorescent moieties can be visualized or otherwise detected and quantitated. Two oligonucleotide probes, each containing a fluorescent moiety, can hybridize to an amplification product at particular positions determined by the complementarity of the oligonucleotide probes to the target nucleic acid sequence. Upon hybridization of the oligonucleotide probes to the amplification product at the appropriate positions, a FRET signal is generated. Hybridization temperatures and times can range from about 35° C. to about 65° C. for about 10 seconds to about 1 minute. Detection of FRET can occur in real-time, such that the increase in an amplification product after each cycle of a PCR assay is detected and, in some embodiments, quantified.

Fluorescent analysis and quantification can be carried out using, for example, a photon counting epifluorescent microscope system (containing the appropriate dichroic mirror and filters for monitoring fluorescent emission in a particular range of wavelengths), a photon counting photomultiplier system, or a fluorometer. Excitation to initiate energy transfer can be carried out with an argon ion laser, a high intensity mercury arc lamp, a fiber optic light source, or another high intensity light source appropriately filtered for excitation in the desired range.

Fluorescent moieties can be, for example, a donor moiety and a corresponding acceptor moiety. As used herein with respect to donor and corresponding acceptor fluorescent moieties, “corresponding” refers to an acceptor fluorescent moiety having an emission spectrum that overlaps the excitation spectrum of the donor fluorescent moiety. The wavelength maximum of the emission spectrum of an acceptor fluorescent moiety typically should be at least 100 nm greater than the wavelength maximum of the excitation spectrum of the donor fluorescent moiety, such that efficient non-radiative energy transfer can be produced there between.

Fluorescent donor and corresponding acceptor moieties are generally chosen for (a) high efficiency Forster energy transfer; (b) a large final Stokes shift (>100 nm); (c) shift of the emission as far as possible into the red portion of the visible spectrum (>600 nm); and (d) shift of the emission to a higher wavelength than the Raman water fluorescent emission produced by excitation at the donor excitation wavelength. For example, a donor fluorescent moiety can be chosen with an excitation maximum near a laser line (for example, Helium-Cadmium 442 nm or Argon 488 nm), a high extinction coefficient, a high quantum yield, and a good overlap of its fluorescent emission with the excitation spectrum of the corresponding acceptor fluorescent moiety. A corresponding acceptor fluorescent moiety can be chosen that has a high extinction coefficient, a high quantum yield, a good overlap of its excitation with the emission of the donor fluorescent moiety, and emission in the red part of the visible spectrum (>600 nm).

Representative donor fluorescent moieties that can be used with various acceptor fluorescent moieties in FRET technology include fluorescein, Lucifer Yellow, B-phycoerythrin, 9-acridineisothiocyanate, Lucifer Yellow VS, 4-acetamido-4′-isothiocyanatostilbene-2,2′-disulfonic acid, 7-diethylamino-3-(4′-isothiocyanatophenyl)-4-methylcoumarin, succinimdyl 1-pyrenebutyrate, and 4-acetamido-4′-isothiocyanatostilbene-2,2′-disulfonic acid derivatives. Representative acceptor fluorescent moieties, depending upon the donor fluorescent moiety used, include LC™-Red 640, LC™-Red 705, Cy5, Cy5.5, Lissamine rhodamine B sulfonyl chloride, tetramethyl rhodamine isothiocyanate, rhodamine x isothiocyanate, erythrosine isothiocyanate, fluorescein, diethylenetriamine pentaacetate, and other chelates of Lanthanide ions (e.g., Europium, or Terbium). Donor and acceptor fluorescent moieties can be obtained from, for example, Molecular Probes, Inc. (Eugene, Oreg.) or Sigma Chemical Co. (St. Louis, Mo.).

Donor and acceptor fluorescent moieties can be attached to probe oligonucleotides via linker arms. The length of each linker arm is important, as the linker arms will affect the distance between the donor and acceptor fluorescent moieties. The length of a linker arm for the purpose of the present invention is the distance in Angstroms (Å) from the nucleotide base to the fluorescent moiety. In general, a linker arm is from about 10 to about 25 Å in length. The linker arm may be of the kind described in WO 84/03285, for example. WO 84/03285 also discloses methods for attaching linker arms to a particular nucleotide base, as well as methods for attaching fluorescent moieties to a linker arm.

An acceptor fluorescent moiety such as an LC™-Red 640-NHS-ester can be combined with C6-Phosphoramidites (available from ABI (Foster City, Calif.) or Glen Research (Sterling, Va.)) to produce, for example, LC™-Red 640-Phosphoramidite. Linkers frequently used to couple a donor fluorescent moiety such as fluorescein to an oligonucleotide include thiourea linkers (FITC-derived, for example, fluorescein-CPG's from Glen Research or ChemGene (Ashland, Mass.)), amide-linkers (fluorescein-NHS-ester-derived, such as fluorescein-CPG from BioGenex (San Ramon, Calif.)), or 3‘-amino-CPG’s that require coupling of a fluorescein-NHS-ester after oligonucleotide synthesis.

Using commercially available real-time PCR instrumentation (e.g., LightCycler™, Roche Molecular Biochemicals, Indianapolis, Ind.), PCR amplification, detection, and quantification of an amplification product can be combined in a single closed cuvette with dramatically reduced cycling time. Since detection and quantification occur concurrently with amplification, real-time PCR methods obviate the need for manipulation of the amplification product, and diminish the risk of cross-contamination between amplification products. Real-time PCR greatly reduces turn-around time and is an attractive alternative to conventional PCR techniques in the clinical laboratory or in the field.

The amount of FRET corresponds to the amount of amplification product, which in turn corresponds to the amount of template nucleic acid present in the sample. Similarly, the amount of template nucleic acid corresponds to the amount of microbial organism present in the sample. Therefore, the amount of FRET produced when amplifying nucleic acid obtained from a biological sample can be correlated to the amount of a microorganism. Typically, the amount of a microorganism in a sample can be quantified by comparing the amount of FRET produced from amplified nucleic acid obtained from known amounts of the microorganism. Accurate quantitation requires measuring the amount of FRET while amplification is increasing linearly. In addition, there must be an excess of probe in the reaction. Furthermore, the amount of FRET produced in the known samples used for comparison purposes can be standardized for particular reaction conditions, such that it is not necessary to isolate and amplify samples from every microorganism for comparison purposes.

Melting curve analysis is an additional step that can be included in a cycling profile. Melting curve analysis is based on the fact that a nucleic acid sequence melts at a characteristic temperature (Tm), which is defined as the temperature at which half of the DNA duplexes have separated into single strands. The melting temperature of a DNA molecule depends primarily upon its nucleotide composition. A DNA molecule rich in G and C nucleotides has a higher Tm than one having an abundance of A and T nucleotides. The temperature at which the FRET signal is lost correlates with the melting temperature of a probe from an amplification product. Similarly, the temperature at which signal is generated correlates with the annealing temperature of a probe with an amplification product. The melting temperature(s) of target probes from an amplification product can confirm the presence or absence of the target-containing species in a sample, and can be used to quantify the amount of a particular target-containing species. For example, a universal probe that hybridizes to a variable region within cpn60 will have a Tm that depends upon the sequence to which it hybridizes. Thus, a universal probe may have a Tm of 70° C. when hybridized to a cpn60 amplification product generated from one microbial organism, but a Tm of 65° C. when hybridized to a cpn60 amplification product generated from a second microbial organism. By observing a temperature-dependent, step-wise decrease in fluorescence of a sample as it is heated, the particular cpn60-containing species in the sample can be identified and the relative amounts of the species in the sample can be determined.

Control samples can be included within each thermocycler run. Positive control samples can amplify a nucleic acid control template (e.g., a nucleic acid other than a target nucleic acid) using, for example, control primers and control probes. Positive control samples also can amplify, for example, a plasmid construct containing a control nucleic acid template. Such a plasmid control can be amplified internally (e.g., within the sample) or in a separate sample run side-by-side with the test samples. Each thermocycler run also should include a negative control that, for example, lacks the target template DNA. Such controls are indicators of the success or failure of the amplification, hybridization and/or FRET reaction. Therefore, control reactions can readily determine, for example, the ability of primers to anneal with sequence-specificity and to initiate elongation, as well as the ability of probes to hybridize with sequence-specificity and for FRET to occur.

As an alternative to FRET, amplification product can be detected using, for example, a fluorescent DNA binding dye (e.g., SYBRGreenI® or SYBRGold® (Molecular Probes)). Upon interaction with an amplification product, such DNA binding dyes emit a fluorescent signal after excitation with light at a suitable wavelength. A double-stranded DNA binding dye such as a nucleic acid intercalating dye also can be used. When double-stranded DNA binding dyes are used, a melting curve analysis usually is performed for confirmation of the presence of the amplification product.

Scheduling Animal Processing

After determining the microbial load and/or microbial profile of the animals, processing can be scheduled by, for example, the manager of the processing facility. As used herein, a “manager” refers to any individual(s) associated with managing the operation and making decisions concerning the animals in such an operation. As used herein, a “manager” can oversee the operations of a growing or processing facility, or a “manager” can be a caretaker, the person responsible for feeding the animals, or the person responsible for slaughtering or preparing the animals for slaughter.

Typically, scheduling of processing includes ranking the animals according to the microbial load and/or microbial profile information and scheduling animals with more desirable characteristics for processing earlier in the day to minimize contamination of processing equipment. For example, animals having the lowest microbial load can be scheduled for processing earlier in the day than animals with higher microbial loads. Alternatively, it may be desirable to process animals with a lower microbial load on one day and animals with a higher microbial load on a different day. Similarly, animals having a more favorable microbial profile (e.g., lower incidence of particular pathogens such as Salmonella or Campylobacter in poultry) or a lower risk profile (e.g., lower total microbial load and lower incidence of pathogens) can be scheduled for processing earlier in the day than animals with a less favorable microbial profile or higher risk profile.

Ranking of the animals according to risk can be integrated with one or more additional factors that can affect animal processing. Additional factors that can be considered include, for example, the number of animals to be processed, weight target of animals, amount of feed remaining at the farm, proximity of the animals to the animal processing facility, and time of delivery of animals to the animal processing facility.

Computer System and Software

The invention provides a system that can be used for scheduling a plurality of animals for processing. Such a system generally includes means for inputting microbial load and/or profile information for a plurality of animals and a processor for correlating microbial load and microbial profile with risk and for scheduling the animals for processing based at least in part on risk. Processers can be contained within computers suitable for use as a personal computer owned and operated by an individual or it can be a server or other computer system used by businesses in their operations. For example, a processer can be a server upon which web pages can be stored and through which the pages are publicly accessible by others. Computers typically include a main unit that contains the essentials for computing: a processor, a primary memory, a secondary memory, ports and connectors for input and output, and circuitry connecting the components to make the computer functional, as is well known in the art. Output from the computer can be displayed through a monitor that is usually connected to the main unit. Output from the computer also can be transmitted to a printer that is connected to the computer. A user may submit information and commands though various input means as is well known. For example, a user may employ a keyboard and/or a pointing device to enter data and commands. The computer can be connected to a public computer network, such as the Internet, or to a private or semi-private network, through a cable. Other means of connecting to public networks are also contemplated and this description is not limited to any connection in particular. A computer for use in the invention can be a portable device similar to, for example, a personal digital assistant (PDA).

A computer typically operates by executing program steps that may be stored in memory or that may be accessible from sources outside the computer. The computer may access information on a storage medium capable of being read in a media reader. For example, the reader may be capable of reading a computer-readable storage medium such as either or both 3.5″ floppy disks or compact disks (CDs). Accessing sources outside the computer can occur via any suitable means, such as e-mail, hardwire connection, wireless network, or cellular network.

As used herein, “means for inputting microbial loads” and “means for inputting profile information” can refer to any method by which microbial loads or profile data are introduced into or obtained by a system of the invention. For example, biological instrumentation can be included in a system of the invention and can be used to obtain and transfer microbial profile data evaluated from a biological sample into the system. Microbial load and profile information also can be directly keyed in using, for example, a keyboard. In addition, microbial load and profile information can be introduced into a system of the invention using a media reader that accesses microbial profile information contained on a floppy disk, CD, or other storage medium. Alternatively, subject microbial profile information can be obtained from a remote source such as a database, wherein the system accesses the database via a network connection.

A “processor for correlating and scheduling” generally refers to a processor that is internal to a system of the invention. Alternatively, a processor for scheduling can be a remote processor such as one accessed over a network connection. A remote processor can be, but is not required to be, at the same remote site at which microbial profile information is accessed.

The invention also provides for a computer-readable storage medium. A computer-readable storage medium can be, for example, a floppy disk, a CD, or other storage medium. A computer-readable storage medium of the invention contains instructions (e.g., a software application) stored thereon for causing a processor to rank a plurality of animals from lowest to highest microbial load or from lowest to highest risk. In one embodiment, the microbial load information of animals is accessed from memory or from a computer-readable storage medium. A computer-readable storage medium of the invention can further include instructions for causing the processor to scheduling processing of the animals based on the rank and one or more additional factors (as described above).

The invention will be further described in the following examples, which do not limit the scope of the invention described in the claims.

### EXAMPLES

### Example 1

### Quantitating Microbial Organisms Using Universal Primers and a Universal Probe

A biological sample is obtained from poultry GIT and genomic DNA is extracted using standard methods (Diagnostic Molecular Microbiology: Principles and Applications (supra)). Real-time PCR is conducted using universal cpn60 primers having the nucleotide sequences set forth in SEQ ID NO:6 and SEQ ID NO:7, and a universal cpn60 probe having the sequence 5′-GACAAAGTCGGTAAAGAAGGCGTTATCA-3′ (SEQ ID NO:8), labeled at the 5′ end with fluorescein (fluorophore; Molecular Probes, Inc.) and at the 3′ end with dabcyl (quencher; (4-(4′-dimethylaminophenylazo)benzoic acid) succinimidyl ester; Molecular Probes, Inc.). This probe binds to a variable region of the cpn60 gene from numerous microbial species; thus the Tm of the probe from an amplification product varies depending upon the nucleotide sequence within the amplification product to which the probe hybridizes.

The PCR reaction contains 3 μL extracted DNA, 1 μM each universal cpn60 primer, 340 nM universal cpn60 probe, 2.5 units Amplitaq Gold DNA polymerase (Applied Biosystems), 0.25 mM each deoxyribonucleotide, 3.5 mM MgCl₂, 50 mM KCl, and 10 mM Tris-HCl, pH 8.0 in a total reaction volume of 50 μL. PCR conditions include an initial incubation at 95° C. for 10 minutes to activate the Amplitaq Gold DNA polymerase, followed by 40 cycles of 30 seconds at 95° C., 60 seconds at 50° C., and 30 seconds at 72° C. Fluorescence is monitored during the 50° C. annealing steps throughout the 40 cycles. After the cycling steps are complete, the melting temperature of the universal probe from the amplification products is analyzed. As the temperature is increased, the universal probe is released from the amplification product from each species' cpn60 sequence at a specific temperature, corresponding to the Tm of the universal probe and the cpn60 sequence of the particular species. The step-wise loss of fluorescence at particular temperatures is used to identify the particular species present, and the loss in fluorescence of each step compared to the total amount of fluorescence correlates with the relative amount of each microorganism.

### Example 2

### Quantification of Microbial Organisms Using Universal Primers and Species-Specific Probes

A biological sample is obtained from poultry GIT and genomic DNA is extracted using standard methods (Diagnostic Molecular Microbiology: Principles and Applications (supra)). Real-time PCR is conducted using universal cpn60 primers having the nucleotide sequences set forth in SEQ ID NO:6 and SEQ ID NO:7, and species-specific probes having the nucleotide sequences 5′-AGCCGTTGCAAAAGCAGGCAAACCGC-3′ (SEQ ID NO:9), 5′-TTGAGCAAATAGTTCAAGCAGGTAA-3′ (SEQ ID NO:10), 5′GCAACTCTGGTTGTTAACACCATGC-3′ (SEQ ID NO:11), 5′TGGAGAAGGTCATCCAGGCCAACGC-3′ (SEQ ID NO:12), and 5′TAGAACAAATTCAAAAAACAGGCAA-3′ (SEQ ID NO:13). These species-specific probes hybridize to cpn60 nucleotide sequences from S. enterica, C. perfringens, E. coli, S. coelicolor, and C. jejuni, respectively. The sequences of the probes are identified by aligning cpn60 cDNA sequences from the five organisms and identifying a sequence that is unique to each particular organism (i.e., a sequence not found in the other organisms). Each of the species-specific probes is labeled with a different fluorescent moiety to allow differential detection of the various species.

The PCR reaction contains 3 μL extracted DNA, 1 μM each universal cpn60 primer, 340 nM universal cpn60 probe, 2.5 units Amplitaq Gold DNA polymerase (Applied Biosystems), 0.25 mM each deoxyribonucleotide, 3.5 mM MgCl₂, 50 mM KCl, and 10 mM Tris-HCl, pH 8.0 in a total reaction volume of 50 μL. PCR conditions include an initial incubation at 95° C. for 10 minutes to activate the Amplitaq Gold DNA polymerase, followed by 40 cycles of 30 seconds at 95° C., 60 seconds at 50° C., and 30 seconds at 72° C. Fluorescence is monitored during the 50° C. annealing steps throughout the 40 cycles, at wavelengths corresponding to the particular moieties on the probes. The amount of fluorescence detected at each of the monitored wavelengths correlates with the amount of each cpn60 amplification product. The amount of each species-specific amplification product is then correlated with the amount of each species of microbe by comparison to the amount of amplification product generated from positive control samples containing nucleic acid isolated from known amounts of each microbial species. Nucleic acids in the positive control samples can be obtained from, for example, E. coli or Salmonella spp.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of managing an animal processing operation, said method comprising:
a) providing biological samples from a plurality of animals;
b) determining microbial load of each said biological sample; and
c) scheduling processing of said plurality of animals based at least in part on said microbial load.

2. The method of claim 1, wherein said animals are birds.

3. The method of claim 2, wherein said birds are chickens or turkeys.

4. The method of claim 1, wherein said animals are pigs.

5. The method of claim 1, wherein said animals are cows.

6. The method of claim 1, wherein said animals are fish.

7. The method of claim 1, wherein microbial load is total number of enteric bacteria.

8. The method of claim 1, wherein scheduling comprises ranking said animals from lowest microbial load to highest microbial load and integrating said ranking with one or more factors selected from the group consisting of number of animals to be processed, weight targets of animals, proximity of animals to animal processing facility, and time of delivery of animals to animal processing facility.

9. The method of claim 1, said method further comprising determining the microbial profile of each said biological sample and scheduling processing of said plurality of animals based at least in part on said microbial load and microbial profile.

10. The method of claim 9, wherein scheduling comprises ranking said animals from lowest risk to highest risk based on said microbial load and microbial profile to provide a risk profile; and integrating said risk profile with one or more factors selected from the group consisting of number of animals to be processed, weight targets of animals, proximity of animals to animal processing facility, and time of delivery of animals to animal processing facility.

11. The method of claim 9, wherein determining the microbial profile comprises histological analysis.

12. The method of claim 9, wherein determining the microbial profile comprises immunological analysis.

13. The method of claim 9, wherein determining the microbial profile comprises genetic fingerprinting.

14. The method of claim 9, wherein determining the microbial profile comprises ribosomal genotyping.

15. The method of claim 9, wherein determining the microbial profile comprises cpn60 genotyping.

16. The method of claim 1, wherein said plurality of animals comprises a plurality of different populations of animals.

17. The method of claim 16, wherein said biological samples comprise pooled samples from each said population of animals.

18. The method of claim 1, wherein said biological samples are gastrointestinal tract samples.

19. The method of claim 18, wherein said gastrointestinal tract samples are digesta.

20. The method of claim 18, wherein said gastrointestinal tract samples are mucous and/or mucosal tissue.

21. The method of claim 18, wherein said gastrointestinal tract samples are feces.

22. The method of claim 1, wherein said biological samples are periodontal samples.

23. The method of claim 1, wherein said biological samples are bodily fluids.

24. The method of claim 23, wherein said bodily fluids are selected from the group consisting of blood, urine, saliva, sputum, and semen.

25. The method of claim 9, wherein said microbial profile is based on taxonomic and phylogenetic identification of microbes in said biological sample.

26. The method of claim 25, wherein said microbes are selected from the group consisting of bacteria, protozoa, and fungi.

27. A computer-readable storage medium having instructions stored thereon for causing a programmable processor to rank a plurality of animals from lowest microbial load to highest microbial load.

28. The computer-readable storage medium of claim 27, further comprising instructions for scheduling processing of said plurality of animals based at least in part on said rank.

29. The computer readable storage medium of claim 27, further comprising instructions for scheduling processing of said plurality of animals based on said rank and one or more factors selected from the group consisting of number of animals to be processed, weight targets of animals, proximity of animals to animal processing facility, and time of delivery of animals to animal processing facility.

30. The computer-readable storage medium of claim 27, further comprising instructions for causing said programmable processor to determine a risk profile for said plurality of animals based on microbial loads and microbial profiles from said plurality of animals and to rank said plurality of animals from lowest risk to highest risk.

31. The computer-readable storage medium of claim 30, further comprising instructions for scheduling processing of said plurality of animals based on said risk profile and one or more factors selected from the group consisting of number of animals to be processed, weight targets of animals, amount of feed, proximity of animals to animal processing facility, and time of delivery of animals to animal processing facility.

32. A system for scheduling a plurality of animals for processing, said system comprising:
a) means for inputting microbial loads of said plurality of animals;
b) means for inputting microbial profiles of said plurality of animals; and
c) a processor for (i) correlating microbial load and microbial profiles with risk and (ii) scheduling said plurality of animals for processing based at least in part on risk.

33. A method of managing an animal processing operation, comprising:
a) providing a risk profile of a plurality of animals; and
b) communicating said risk profile to a manager of said animal processing operation.


SEQUENCE LISTING (13 sequences)

SEQ ID NO: 1 - DNA, 1620 bp, Clostridium perfringens
atggctaaaacattattattcggtgaagaagcaagaagatctatgcaagcgggtgtagataaattagctaacactgttaaggttacattaggaccaaaaggaagaaatgttattttagataaaaaatttggatcaccattaataacaaatgatggggttacaatagcaagagaaattgaacttgaagatgcttatgaaaatatgggagctcaacttgtaaaagaagtagctacaaagactaatgatgtggcaggagatggaactactacagctaccttattagctcaagcaattataagagaaggattaaaaaatgtaacagcaggggcaaatcctatattaataagaaatggaattaaaactgcagttgaaaaagctgtagaggaaatacaaaaaatttctaagcctgtaaatggaaaagaagacatagctagagttgctgcaatttcagcggctgatgaaaaaattggtaagctaattgcagatgctatggaaaaggtaggaaatgaaggcgttataactgtagaagaatctaaatcaatgggaactgagttagatgttgttgaaggtatgcaatttgatagaggatatgtatcagcttatatggttactgatactgaaaaaatggaagctgttttagataatccattagtattaataacagataagaaaataagcaatatacaagatttattaccattacttgagcaaatagttcaagcaggtaaaaaacttttaataatagctgatgatatagaaggcgaagctatgacaacattagttgttaataaattaagaggaacatttacttgtgttggagttaaagcacctggatttggtgatagaagaaaagaaatgttacaagatatagctactttaacaggtggcgttgttatatctgatgaagtaggcggagatttaaaagaagctacattagatatgcttggagaagctgaaagtgttaaggtaactaaagaaagtactacaatagttaatggaagaggaaactcagaagagattaaaaatagagttaaccaaataaaattacaattagaagctactacttctgaatttgacaaagaaaaattacaagaaagattagctaaattagcaggtggggttgcagtagttaaggttggagctgccactgaaacagagcttaaggaaagtaagctaagaatagaggatgctttagcagctacaaaggcagctgttgaagaaggaatagttccaggtggtggaacagcttacgtaaatgtaataaatgaagttgcaaaattaacctctgatattcaagatgaacaagttggtataaatataattgtaagatctttagaagaacctatgagacaaatagctcataatgcaggactagaaggttcagttataatagaaaaagttaaaaatagtgatgcaggtgtaggatttgatgctttaagaggagaatataaagatatgattaaagctggaatagttgatccaactaaggttacaagatcagctcttcaaaatgcagcatcagtagcatcaacattcttaacaacagaggctgctgtagcagatattccagaaaaagaaatgcctcaaggcgcaggtatgggaatggacggaatgtactaa

SEQ ID NO: 2 - DNA, 1647 bp, Escherichia coli
atggcagctaaagacgtaaaattcggtaacgacgctcgtgtgaaaatgctgcgcggcgtaaacgtactggcagatgcagtgaaagttaccctcggtccaaaaggccgtaacgtagttctggataaatctttcggtgcaccgaccatcaccaaagatggtgtttccgttgctcgtgaaatcgaactggaagacaagttcgaaaatatgggtgcgcagatggtgaaagaagttgcctctaaagcaaacgacgctgcaggcgacggtaccaccactgcaaccgtactggctcaggctatcatcactgaaggtctgaaagctgttgctgcgggcatgaacccgatggacctgaaacgtggtatcgacaaagcggttaccgctgcagttgaagaactgaaagcgctgtccgtaccatgctctgactctaaagcgattgctcaggttggtaccatctccgctaactccgacgaaaccgtaggtaaactgatcgctgaagcgatggacaaagtcggtaaagaaggcgttatcaccgttgaagacggtaccggtctgcaggacgaactggacgtggttgaaggtatgcagttcgaccgtggctacctgtctccttacttcatcaacaagccggaaactggcgcagtagaactggaaagcccgttcatcctgctggctgacaagaaaatctccaacatccgcgaaatgctgccggttctggaagctgttgccaaagcaggcaaaccgctgctgatcatcgctgaagatgtagaaggcgaagcgctggcaactctggttgttaacaccatgcgtggcatcgtgaaagtcgctgcggttaaagcaccgggcttcggcgatcgtcgtaaagctatgctgcaggatatcgcaaccctgactggcggtaccgtgatctctgaagagatcggtatggagctggaaaaagcaaccctggaagacctgggtcaggctaaacgtgttgtgatcaacaaagacaccaccactatcatcgatggcgtgggtgaagaagctgcaatccagggccgtgttgctcagatccgtcagcagattgaagaagcaacttctgactacgaccgtgaaaaactgcaggaacgcgtagcgaaactggcaggcggcgttgcagttatcaaagtgggtgctgctaccgaagttgaaatgaaagagaaaaaagcacgcgttgaagatgccctgcacgcgacccgtgctgcggtagaagaaggcgtggttgctggtggtggtgttgcgctgatccgcgtagcgtctaaactggctgacctgcgtggtcagaacgaagaccagaacgtgggtatcaaagttgcactgcgtgcaatggaagctccgctgcgtcagatcgtattgaactgcggcgaagaaccgtctgttgttgctaacaccgttaaaggcggcgacggcaactacggttacaacgcagcaaccgaagaatacggcaacatgatcgacatgggtatcctggatccaaccaaagtaactcgttctgctctgcagtacgcagcttctgtggctggcctgatgatcaccaccgaatgcatggttaccgacctgccgaaaaacgatgcagctgacttaggcgctgctggcggtatgggcggcatgggtggcatgggcggcatgatgtaa

SEQ ID NO: 3 - DNA, 1626 bp, Staphylococcus coelicolor
atggcgaagatcctgaagttcgacgaggacgcccgtcgcgccctcgagcgcggcgtcaacaagctcgccgacaccgtgaaggtgacgatcggccccaagggccgcaacgtcgtcatcgacaagaagttcggcgcccccaccatcaccaacgacggcgtcaccatcgcccgcgaggtcgaggtcgaggacccgtacgagaacctcggcgcccagctggtgaaggaggtggcgaccaagaccaacgacatcgcgggtgacggcaccaccaccgccaccgtgctcgcccaggcgctcgtgcgcgagggcctgaagaacgtcgccgccggtgcctccccggcgctgctgaagaagggcatcgacgcggccgtcgccgccgtgtcggaagaccttctcgccaccgcccgcccgatcgacgagaagtccgacatcgccgccgtggccgcgctgtccgcccaggaccagcaggtcggcgagctgatcgccgaagcgatggacaaggtcggcaaggacggtgtcatcaccgtcgaggagtccaacaccttcggtctggagctggacttcaccgagggcatggccttcgacaagggctacctgtcgccgtacttcgtgacggaccaggagcgcatggaggccgtcctcgacgacccgtacatcctgatcaaccagggcaagatctcctccatcgcggacctgctgccgctgctggagaaggtcatccaggccaacgcctccaagccgctgctgatcatcgccgaggacctggagggcgaggcgctctccaccctcgtcgtcaacaagatccgcggcaccttcaacgcggtggccgtcaaggcccccggcttcggcgaccgccgcaaggcgatgctgcaggacatggccgtcctcaccggcgccacggtcatctccgaggaggtcggcctcaagctcgaccaggtcggcctcgaggtgctcggcaccgcccgccgcatcaccgtcaccaaggacgacaccacgatcgtcgacggtgccggcaagcgcgacgaggtccagggccgcatcgcccagatcaaggccgagatcgagaacacggactccgactgggaccgcgagaagctccaggagcgcctcgcgaagctggccggcggcgtgtgcgtgatcaaggtcggcgccgccaccgaggtggagctgaaggagcgcaagcaccgtctggaggacgccatctccgcgacccgcgccgcggtcgaggagggcatcgtctccggtggtggctccgcgctggtccacgccgtcaaggtgctcgagggcaacctcggcaagaccggcgacgaggccaccggtgtcgcggtcgtccgccgcgccgcggtcgagccgctgcgctggatcgccgagaacgccggcctggagggttacgtcatcacctccaaggtcgccgacctcgacaagggccagggcttcaacgccgccaccggcgagtacggcgacctggtcaaggccggcgtcatcgacccggtgaaggtcacccgctccgccctggagaacgccgcctccatcgcctccctcctgctgacgaccgagaccctggtcgtcgagaagaaggaagaggaagagccggccgccggtggccacagccacggccactcccactga

SEQ ID NO: 4 - DNA, 1638 bp, Campylobacter jejuni
atggcaaaagaaattattttttcagatgaagcaagaaataaactttatgagggcgttaaaaaacttaatgacgcggtaaaagtaactatggggccaagaggacgcaatgttttaatccaaaaaagctttggtgctccaagcattactaaagatggcgtaagtgttgctaaagaagtagagcttaaagatagtcttgaaaatatgggtgcttcactcgtaagagaagtagcgagtaaaacagctgatcaagcaggcgatggaacaactactgcaacggttttagctcatgcaattttcaaagaaggtttaagaaatatcacagcaggtgcaaatcctatcgaggtaaaacgcggtatggataaagcttgcgaagctatagtagcagaacttaaaaaactttctcgcgaagtaaaagataaaaaagaaattgcacaagttgctacaatctcagccaactctgatgaaaaaatcggaaatttaatcgctgatgctatggaaaaagtgggcaaagatggtgttatcactgttgaagaggcaaaatcaatcaatgatgaattaaatgtagttgaaggtatgcaatttgacagaggttatttaagcccttattttatcactaatgcagaaaaaatgacagtagagctttcaagcccttatatcctgctttttgataaaaaaattacaaatttaaaagatttattaccggttttagaacaaattcaaaaaacaggcaaaccacttttaattatcgctgaagatattgaaggtgaagcgcttgcaactttggttgtaaataaacttcgcggtgttcttaatatttcagcagtgaaagctccaggttttggcgatagaagaaaagctatgcttgaagatatagcgattttaacaggtggagaagtgatttctgaagaacttggaagaactcttgaaagtgcgactatacaagatcttggacaagcttctagtgtaatcatcgataaagacaatacaaccatagtaaatggtgcaggcgaaaaagcaaatattgatgcgagagtcaatcaaatcaaagcacaaattgctgaaacaacttcagattatgacagagaaaaattacaagaaagacttgcaaaattaagtggtggtgttgcagttattaaagtaggtgcagcaactgaaactgaaatgaaagagaaaaaagatcgcgttgacgatgctttaagcgctactaaagcagcagttgaagaaggtatagtaattggtggtggtgcagcgcttatcaaagcaaaagctaaaatcaaacttgatctacagggtgatgaagcaattggcgcagctatcgttgaaagagctttaagagcacctttaagacaaattgctgaaaatgcaggatttgatgcaggtgtggttgtaaatagcgtagaaaatgctaaagatgaaaacacaggatttgatgctgcaaaaggtgaatatgttaatatgcttgaaagtggaattatcgatcctgttaaagtagaaagagtagctttactcaatgcagtttctgtagctagtatgcttttaaccacagaagcaacaattagtgaaattaaagaagataaacctactatgccagatatgagcggtatgggaggaatgggtggcatgggcggaatgatgtaa

SEQ ID NO: 5 - DNA, 1647 bp, Salmonella enterica
atggcagctaaagacgtaaaattcggtaacgacgctcgtgtgaaaatgctgcgcggcgtaaacgtactggcagatgcagtgaaagtaaccctcggtccgaaaggccgtaacgtggttctggataaatctttcggtgcgccgactatcactaaagatggtgtttccgtagcgcgtgaaatcgagctggaagacaagtttgaaaacatgggcgcgcagatggtgaaagaagttgcctctaaagcgaacgacgctgcaggcgacggcaccaccaccgcgaccgtactggcgcagtccatcattaccgaaggcctgaaagccgttgctgcgggcatgaacccgatggacctgaaacgtggtatcgacaaagcggttgctgctgcggttgaagagctgaaggcgctgtccgtaccgtgctccgactctaaagcgattgctcaggtaggtactatttccgctaactccgacgaaaccgtaggtaaactgatcgcggaagcgatggataaagtcggtaaagaaggcgtcatcactgttgaagacggtaccggtctgcaggacgaactggacgtggttgaaggtatgcagtttgaccgcggctacctgtctccttacttcatcaacaagccggaaactggcgcagtagaactggaaagcccgttcatcctgctggctgataagaaaatctccaacatccgcgaaatgctgccggttctggaagccgttgcaaaagcaggcaaaccgctgctgatcatcgctgaagatgttgaaggcgaagcgctggctaccctggtagtgaacaccatgcgtggcatcgtgaaagtggctgcggttaaagcaccgggcttcggcgatcgtcgtaaggcgatgctgcaggatatcgctaccctgaccggcggtaccgtaatctctgaagagatcggtatggagctggaaaaagcaaccctggaagacctgggtcaggcgaaacgtgttgtgatcaacaaagacaccaccaccatcattgatggcgtgggtgaagaagctgccatccagggccgtgttgctcagatccgtcagcagattgaagaagcgacctccgactacgatcgtgaaaaactgcaggagcgcgtagcgaaactggcaggcggcgttgcggtaatcaaagttggcgctgcgaccgaagttgaaatgaaagagaagaaagcccgcgttgaagatgccctgcacgcgacccgtgctgcggtagaagaaggcgtggttgctggtggtggcgttgcgctgatccgcgttgcttctaaaattgctgacctgaaaggccagaacgaagaccagaacgtgggtatcaaagttgcgctgcgcgcaatggaagctccgctgcgtcagatcgtgctgaactgcggcgaagagccgtctgttgtcgctaacaccgttaaaggcggcgacggtaactacggttacaacgcagcaactgaagaatacggcaacatgatcgatatgggtatcttggacccaaccaaagttacccgttctgcgctgcaatacgcggcttctgtggctggtctgatgatcactaccgagtgcatggtgaccgacctgccgaaaagcgatgctcctgatttaggcgctgctggcggcatgggtggtatgggtggtatgggcggcatgatgtaa

SEQ ID NO: 6 - DNA, 26 bp, Artificial Sequence
  Primer
gannnngcnggngayggnacnacnac

SEQ ID NO: 7 - DNA, 26 bp, Artificial Sequence
  Primer
yknykntcnccraanccnggngcytt

SEQ ID NO: 8 - DNA, 28 bp, Artificial Sequence
  Probe
gacaaagtcggtaaagaaggcgttatca

SEQ ID NO: 9 - DNA, 26 bp, Artificial Sequence
  Probe
agccgttgcaaaagcaggcaaaccgc

SEQ ID NO: 10 - DNA, 25 bp, Artificial Sequence
  Probe
ttgagcaaatagttcaagcaggtaa

SEQ ID NO: 11 - DNA, 25 bp, Artificial Sequence
  Probe
gcaactctggttgttaacaccatgc

SEQ ID NO: 12 - DNA, 25 bp, Artificial Sequence
  Probe
tggagaaggtcatccaggccaacgc

SEQ ID NO: 13 - DNA, 25 bp, Artificial Sequence
  Probe
tagaacaaattcaaaaaacaggcaa